(19) United States
(12) Patent Application Publication
(10) Pub. No.: US 2025/0248935 A1
(43) Pub. Date: Aug. 7, 2025

(54) SYSTEMS AND METHODS FOR ON-DEMAND CLEAVAGE OF HYDROGELS

(71) Applicant: Boston Scientific Scimed, Inc., Maple Grove, MN (US)

(72) Inventors: Rick Dorn, Plymouth, MN (US); Nicolas Ball-Jones, Minneapolis, MN (US); Joseph Thomas Delaney, JR., Minneapolis, MN (US)

(73) Assignee: Boston Scientific Scimed, Inc., Maple Grove, MN (US)

(21) Appl. No.: 19/039,348

(22) Filed: Jan. 28, 2025

Related U.S. Application Data
(60) Provisional application No. 63/549,171, filed on Feb. 2, 2024.

(51) Int. Cl. A61K 9/06; A61K 47/54; A61K 47/60; C08F 8/50

(52) U.S. Cl. CPC A61K 9/06; A61K 47/545; A61K 47/60; C08F 8/50; C08F 2810/20; C08F 2810/50

(57) ABSTRACT

In some aspects, the present disclosure pertains to kits for delivering and degrading a hydrophilic polymer hydrogel. The kits comprise (a) a hydrogel delivery system configured to deliver a hydrophilic polymer hydrogel to a subject, the hydrophilic polymer hydrogel comprising hydrophilic polymer chains that are crosslinked by crosslinks that comprise immolative linkers or by crosslinks that contain reversible covalent linkages that incorporate a reactive dimeric linker and (b) a hydrogel cleavage system configured to deliver a cleavage composition to the subject, wherein the cleavage composition contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks. Other aspects of the present disclosure include methods of treatment using such kits.

### CROSS-REFERENCE TO RELATED APPLICATIONS

The application claims the benefit of U.S. Provisional Patent Application Ser. No. 63/549,171 filed on Feb. 2, 2024, the disclosure of which is incorporated herein by reference.

### FIELD

The present disclosure relates to systems and methods for on-demand cleavage of hydrogels. Such systems and methods are useful, for example, in removal of hydrogels from patients.

### BACKGROUND

Injectable hydrogels are an emerging class of materials having a variety of medical uses. As one specific example, injectable hydrogels have been used to create or maintain space between tissues in order to reduce side effects of off-target radiation therapy. The hydrogel creates a space between the rectum and the prostate, moving the rectum away from the treatment region. This can help to reduce radiation exposure to the rectum and/or provide other desirable benefits.

The use of a hydrogel-based perirectal spacer material in conjunction with prostate radiation therapy is illustrated schematically in FIGS. 1A and 1B. FIG. 1A illustrates a cross-section of the human male anatomy including the prostate 110 and rectal wall 112. When the prostate is treated using radiation therapy there is a higher dose region 114 adjacent to the prostate, which is subjected to high doses of radiation, becoming a lower dose region 116 as one proceeds further from the prostate 110. As illustrated in FIG. 1B, a spacing material 118 can be injected between the prostate 110 and the rectal wall 112, which can push the rectal wall from a higher dose region 114 to a lower dose region 116, thereby reducing injury to the rectal wall.

SpaceOAR® and SpaceOAR Vue® are hydrogels that rapidly form crosslinks in vivo. They are based on multi-arm polyethylene (PEG) polymers having a polyol residue core functionalized with succinimidyl glutarate (SG) activated ester end groups. Above a specific pH, the SG groups will rapidly react with a trilysine crosslinker in vivo to form a hydrogel. The hydrogels break down in-vivo over the course of ca. 6-9 months before they are completely expelled. The breakdown occurs primarily through the hydrolysis of the ester linkages on the glutarate groups. FIG. 2 shows a hydrogel (210) which contains a residue of the multi-arm PEG having a polyol residue core (Core) functionalized with SG end groups and a residue of the trilysine. Upon exposure to water (H₂O) the ester linkage is hydrolyzed breaking down the hydrogel into a hydroxyl-terminated PEG (212) (where only a single arm is shown) and a (3-carboxylpropyl)amide-terminated-lysine residue (214) (where only a single side group residue is shown).

The development of hydrogels that demonstrate triggered/on-demand degradation opens up the door for many medical applications, including hydrogel-based perirectal spacing, among many others, where the hydrogel can persist only until the procedure/therapy is complete or it is otherwise desirable to remove the hydrogel.

### SUMMARY

In some aspects, the present disclosure pertains to kits for delivering and degrading a hydrophilic polymer hydrogel. The kits comprise (a) a hydrogel delivery system configured to deliver a hydrophilic polymer hydrogel to a subject, the hydrophilic polymer hydrogel comprising hydrophilic polymer chains that are crosslinked by crosslinks that comprise immolative linkers or by crosslinks that contain reversible covalent linkages that incorporate a reactive dimeric linker and (b) a hydrogel cleavage system configured to deliver a cleavage composition to the subject, wherein the cleavage composition contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks.

In some embodiments, the hydrophilic polymer hydrogel comprises the hydrophilic polymer chains that are crosslinked by crosslinks that comprise immolative linkers and the hydrogel cleavage composition contains the singly reactive molecule that acts to break the crosslinks at the position of the immolative linkers within the crosslinks. In some of these embodiments, the crosslinks comprise imidosydnone groups as immolative linkers and the singly reactive molecule is a strained alkyne reactive molecule. In some of these embodiments, the crosslinks comprise 1-(methyloxidoamino) cyclooctene groups as immolative linkers and the singly reactive molecule is a diboron molecule. Other combinations of immolative linker and singly reactive molecules are described below.

In some embodiments, the hydrophilic polymer hydrogel comprises the hydrophilic polymer chains that are crosslinked by crosslinks that contain reversible covalent linkages that incorporate a reactive dimeric linker and the hydrogel cleavage composition contains the singly reactive molecule that acts to break the crosslinks at the position of the dimeric linkers within the crosslinks. In some of the embodiments, each of the reversible covalent linkages each comprises two thioester groups, the singly reactive molecule is a thiol molecule, and the reactive dimeric linker is a bis-thiol linker. Other combinations of reversible covalent linkages, singly reactive molecules and reactive dimeric linkers are described below.

In some embodiments, which can be used in conjunction with any of the above aspects and embodiments, the hydrogel cleavage system comprises a syringe barrel that contains the cleavage composition. In some of these embodiments, the hydrogel cleavage system further comprises a needle, a flexible tube, or both, and the cleavage syringe system is configured for coupling to the needle, the flexible tube, or both.

In some embodiments, which can be used in conjunction with any of the above aspects and embodiments, the hydrogel delivery system comprises a preformed hydrophilic polymer hydrogel. In some of these embodiments, the preformed hydrophilic polymer hydrogel comprises particles of the hydrophilic polymer hydrogel. In some of these embodiments, the particles of the hydrophilic polymer hydrogel are prepackaged in a delivery syringe system.

In some embodiments, which can be used in conjunction with any of the above aspects and embodiments, the hydrogel delivery system comprises a first composition comprising a multifunctional crosslinker and a second composition comprising a multi-arm polymer having polymer chains that are reactive with the multifunctional crosslinker. In some of these embodiments, the hydrogel delivery system is configured to form a mixture of the first and second compositions and to deliver the mixture to a subject whereupon the first and second compositions form said crosslinks with one another. In some of these embodiments, the hydrogel delivery system comprises a delivery syringe system that is configured to form a mixture of the first and second compositions and to deliver the mixture to a subject whereupon the first and second compositions form said crosslinks with one another. In some of these embodiments, the delivery syringe system comprises a dual barrel syringe device for creating and delivering the mixture of the first and second compositions.

In some aspects, the present disclosure provides methods that comprise (a) delivering a hydrophilic polymer hydrogel to a subject, the hydrophilic polymer hydrogel comprising hydrophilic polymer chains that are crosslinked by crosslinks that comprise immolative linkers or by crosslinks that contain reversible covalent linkages that incorporate reactive dimeric linkers and (b) delivering a cleavage composition to the subject such that the cleavage composition contacts the hydrophilic polymer hydrogel, wherein the cleavage composition contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks.

In some embodiments, the methods comprise delivering the hydrophilic polymer hydrogel to the subject via a delivery syringe system coupled to a needle; disconnecting the delivery syringe system from the needle; attaching a cleavage syringe system to the needle, the cleavage syringe system including the cleavage composition disposed in a syringe barrel; and injecting the cleavage composition into the subject.

In some embodiments, which can be used in conjunction with any of the above aspects and embodiments, the methods comprise observing a complication associated with the delivering of the hydrophilic polymer hydrogel to the subject prior to injecting the hydrogel cleavage material into the subject.

In some embodiments, which can be used in conjunction with any of the above aspects and embodiments, the methods further comprise delivering a replacement hydrophilic polymer hydrogel into the subject.

The above and other aspects, embodiments, features and benefits of the present disclosure will be readily apparent from the following detailed description.

### BRIEF DESCRIPTION OF THE DRAWINGS

FIGS. 1A and 1B schematically illustrate the use of a hydrogel-based perirectal spacer material in conjunction with prostate radiation therapy, in accordance with the prior art.

FIG. 2 schematically illustrates a process whereby a hydrogel is hydrolyzed, in accordance with the prior art.

FIG. 3A schematically illustrates a process for forming boc-protected 4-imidosyndonebenzoic acid chloride, in accordance with an embodiment of the present disclosure.

FIG. 3B schematically illustrates a process for forming an imidosydnone-functionalized trilysine-derived crosslinker, in accordance with an embodiment of the present disclosure.

FIG. 3C schematically illustrates a process for forming a succinimidyl-ester-terminated multi-arm polymer, in accordance with an embodiment of the present disclosure.

FIG. 3D schematically illustrates a process for forming a hydrophilic polymer hydrogel from the imidosydnone-functionalized trilysine-derived crosslinker of FIG. 3B and the succinimidyl-ester-terminated multi-arm polymer of FIG. 3C, in accordance with an embodiment of the present disclosure.

FIG. 3E schematically illustrates a process for cleaving crosslinks in the hydrophilic polymer hydrogel of FIG. 3D by contact with a strained alkyne, in accordance with an embodiment of the present disclosure.

FIG. 3F schematically illustrates a process for forming an imidosydnone-functionalized trilysine-derived crosslinker, in accordance with a further embodiment of the present disclosure.

FIG. 4A schematically illustrates a process for forming a cyclooctyne-terminated multi-arm polymer, in accordance with an embodiment of the present disclosure.

FIG. 4B schematically illustrates a process for forming an N-methylhydroxylamine-functional lysine-derived crosslinker, in accordance with an embodiment of the present disclosure.

FIG. 4C schematically illustrates a process for forming a hydrophilic polymer hydrogel from the cyclooctyne-terminated multi-arm polymer of FIG. 4A and the N-methylhydroxylamine-functional lysine-derived crosslinker of FIG. 4B, in accordance with an embodiment of the present disclosure.

FIG. 4D schematically illustrates a process for cleaving crosslinks in the hydrophilic polymer hydrogel of FIG. 4C by contact with a diboron compound, in accordance with an embodiment of the present disclosure.

FIG. 5 is a schematic depiction of a region of the human body.

FIG. 6 depicts the placement of a hydrophilic polymer hydrogel spacer in the region of the human body depicted in FIG. 5.

FIG. 7 depicts the delivery of a cleavage composition to the hydrophilic polymer hydrogel spacer of FIG. 6.

FIG. 8 depicts the region of the human body of FIG. 7 after degradation of the hydrophilic polymer hydrogel spacer.

FIG. 9 schematically illustrates a pre-loaded syringe that contains an injectable cleavage composition, in accordance with an embodiment of the present disclosure.

FIG. 10 schematically illustrates a delivery device for delivering a hydrophilic polymer hydrogel, in accordance with an embodiment of the present disclosure.

FIG. 11 schematically illustrates a delivery device for forming a hydrophilic polymer hydrogel in situ, in accordance with an embodiment of the present disclosure.

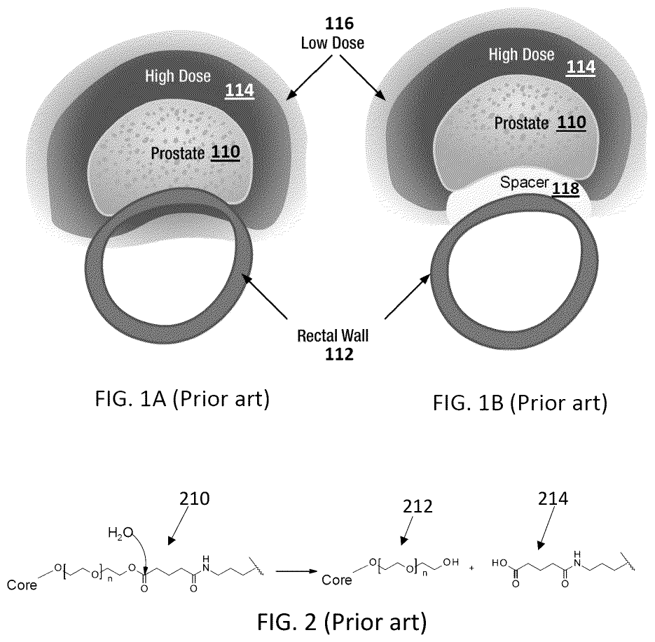

### DETAILED DESCRIPTION

The present disclosure pertains to methods of degrading a hydrophilic polymer hydrogel in situ (i.e., within a subject).

In some aspects, the hydrophilic polymer hydrogel comprises crosslinks between hydrophilic polymer chains within the hydrophilic polymer hydrogel, which crosslinks contain immolative linkers, and the method comprises contacting the hydrophilic polymer hydrogel with a cleavage composition that contains a singly reactive molecule, which acts to break the crosslinks at the position of the immolative linkers within the crosslinks.

In other aspects, the hydrophilic polymer hydrogel comprises crosslinks between hydrophilic polymer chains within the hydrophilic polymer hydrogel, which crosslinks contain reversible covalent linkages that incorporate reactive dimeric linkers, and the method comprises contacting the hydrophilic polymer hydrogel with a cleavage composition that contains a singly reactive molecule, which acts to break the crosslinks at the position of the dimeric linkers within the crosslinks.

Contacting may include, for example, applying the cleavage composition onto a surface of the hydrophilic polymer hydrogel, injecting the cleavage composition into the hydrophilic polymer hydrogel, and so forth.

Such hydrophilic polymer hydrogels may be formed in situ within a subject or may be formed ex vivo and subsequently delivered to a subject.

Preferred subjects include mammalian subjects, particularly human subjects.

As used herein, a “hydrogel” is a crosslinked polymer that contains water or can absorb water but does not dissolve when placed in water.

As used herein an “immolative linker” is defined as a transient covalent bond that can be cleaved through the addition of a triggering molecule.

As used herein a “singly reactive compound” is defined as a compound that will only undergo one reaction with a desired substrate.

With regard to hydrogels having crosslinks that contain reversible covalent linkages that are based on reactive dimeric linkers, a general schematic representation is provided here below, which shows two reversible covalent linkages, represented by X-Y. The covalent linkages. X-Y, reversibly break down resulting (a) a dimeric reactive linker, represented by X-B-X, where B represents a polymer chain and where X represents a reactive group attached to the polymer chain, B, and (b) two polymer chains, represented by AY, where Y represents a reactive group attached to a polymer chain, represented by A, that forms reversible covalent bonds with the reactive X groups of the dimeric reactive linker X-B-X. When a singly reactive molecule, represented by X-D, is introduced, the singly reactive molecule can react with Y groups of each of the AY polymer chains to form further polymer chains, represented by A-Y-X-D, which do not reversibly react with the dimeric reactive linker, X-B-X. In doing so, the singly reactive molecule, X-D, outcompetes the reverse reaction of the dimeric reactive linker, X-B-X, for the polymer chains, AY, which would otherwise reform the reversible covalent linkages. The overall result of this process is that the singly reactive molecule, XD, acts to cleave the reversible covalent linkages.

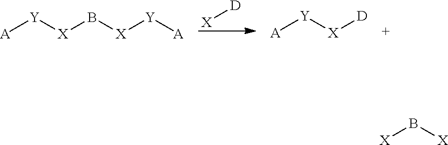

Several specific examples follow. In a first specific example, a crosslink that contains two thioester linkages as reversible covalent linkages is shown being cleaved with a singly reactive molecule, cysteine, or another S-H containing molecule, to yield two polymer chains, each having a thioester group, and a telechelic dithiol reactive linker having two-SH groups:

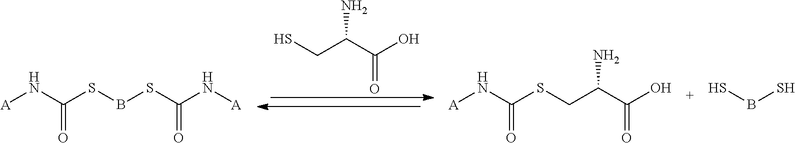

In another specific example, a crosslink that contains two disulfide linkages as reversible covalent linkages is shown being cleaved by a singly reactive molecule, such as cysteine, or another thiol containing small molecule, to yield two polymer chains, each with a disulfide group and a dithiol dimeric reactive linker having two-SH groups:

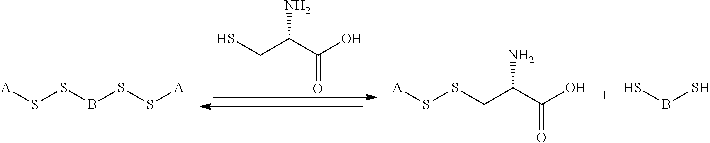

In another specific example, a crosslink that contains two linkages composed of disuccinimide as reversible covalent linkages is shown which reversibly break down into a bismaleimide type reactive linker and two polymer chains, each containing a furyl group. The polymer chains containing the furyl group then react with a singly reactive molecule, poly(N-malimide) to form two polymer chains, each with a succinimide group:

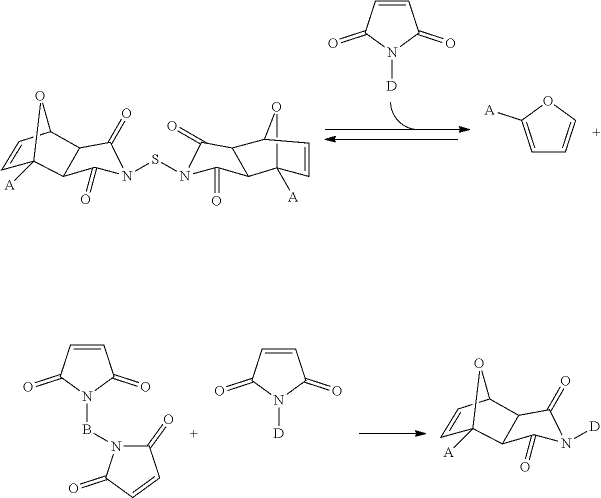

In another specific example, a crosslink that contains two borate ester linkages as reversible covalent linkages is shown which reversibly breaks down into a dimeric reactive linker containing two vicinal diol groups, and two polymer chains, each containing a boronic acid group. The polymer chains then react with a singly reactive molecule, containing a vicinal diol, to form two polymer chains, each with borate ester groups:

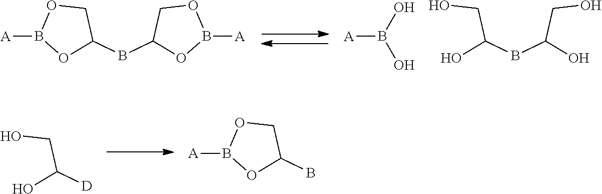

In another specific example, a crosslink that contains two linkages as reversible covalent linkages is shown which reversibly breaks down into a dimeric reactive linker containing two primary amine groups and two polymer chains, each containing an aldehyde group. The polymer chains then react with a singly reactive molecule, for example, lysine, or another primary amine containing small molecule, to form two polymeric chains:

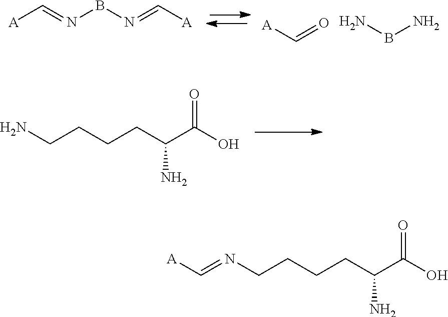

Turning now to hydrogels having crosslinks that contain immolative linkers, several schematic representations of crosslinks between polymer chains follow, which show an immolative linker disposed between two polymer chains, each represented by the letter A, within a crosslinked hydrogel. As will be appreciated from the description to follow, in various embodiments, instead of a single immolative linker as schematically represented, two immolative linkers may be disposed between the chains, with one immolative linker attached to each polymer chain, which immolative linkers are linked to one another through a crosslinker residue. The schematic representations also illustrate the introduction of a singly reactive molecule, which acts to cleave the immolative linker and therefore cleaves the polymer chains from one another.

In one example, a crosslink that contains an imidosydnone group as an immolative linker is shown being cleaved by a strained alkyne reactive molecule to yield one polymer chain with a primary amide group and another polymer chain with a tricyclic group:

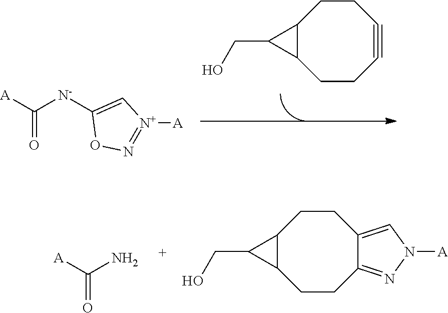

In another example, a crosslink that contains a 1-(methyloxidoamino) cyclooctene group as an immolative linker is shown being cleaved by a diboron reactive molecule to yield one polymer chain with a hydroxyl group and another polymer chain with an iminium group:

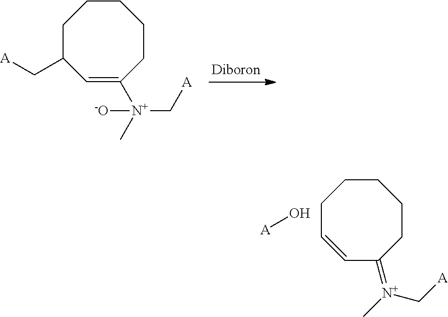

In another example, a crosslink that contains a silyl diether group as an immolative linker, where R is an alkane, cycloalkane, or aromatic side chain is shown being cleaved by a singly reactive molecule, aminophenylethyl trifluoroborate to yield two polymer chains with hydroxyl groups:

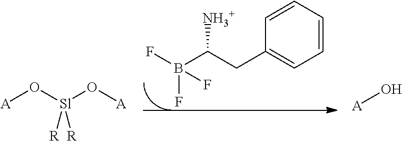

In another example, a crosslink that contains an immolative linker is shown being cleaved by a singly reactive molecule, benzene-1,2-dithiol, to yield one polymer chain with an amine or alcohol group and another polymer chain with a dithiobenzene incorporated into the final aromatic end group of the polymer chain:

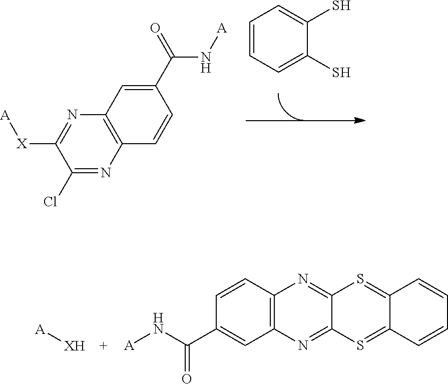

In another example, a crosslink that contains a tetrazine group as an immolative linker is shown being cleaved by a singly reactive molecule, such as a derivatized transcyclooctene, where R is an alkyl group or a 3-hydroxy-cyclooct-1-yne, to yield one polymer chain with a primary amine group and another polymer chain with a pyridazine group:

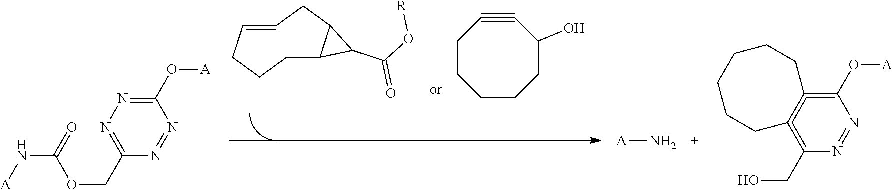

In another example, a crosslink that contains a 4-azidobenzene group as an immolative linker is shown being cleaved by a singly reactive molecule, such as (E)-cyclooct-4-enol, or through reaction with phenyl-2-carboxyl diphenylphosphine to yield one polymer chain with a carboxylate group and another polymer chain with an oxidized aromatic group bound to an amide:

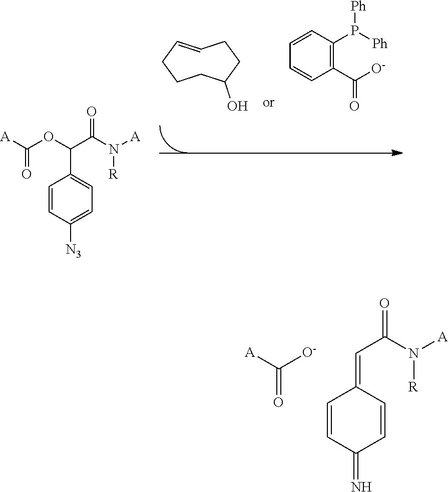

In another example, a crosslink that contains a transcyclooctene group as an immolative linker is shown being cleaved by a singly reactive molecule, such as a tetrazine derivative, where R1 and R2 are alkyl functional groups, to yield one polymer chain with a primary amine group and another polymer chain with a pyridazine group:

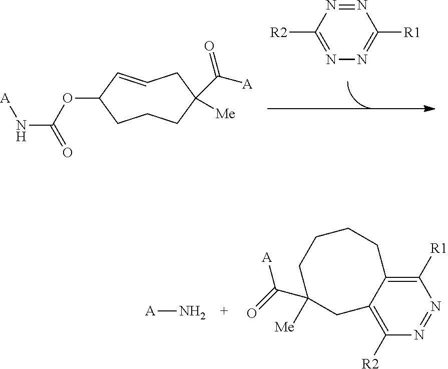

In another example, a crosslink that contains an aryl enol ether group as an immolative linker is shown being cleaved by a singly reactive molecule, such as a tetrazine derivative, where R1 and R2 are alkyl functional groups, to yield one polymer chain with a hydroxyl group and another polymer chain with an alpha beta unsaturated cyclic ketone:

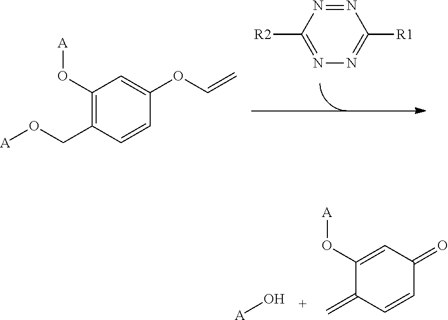

In another example, a crosslink that contains an 1,1 azide ether group as an immolative linker is shown being cleaved by a singly reactive phosphine derivative where R is alkyl or aryl, to yield one polymer chain with an aldehyde group and another polymer chain with a hydroxyl group:

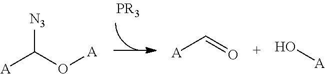

Polymer chains for use herein, including those schematically represented in the preceding schemes with the letter A, may be selected from any of a variety of synthetic, natural, or hybrid synthetic-natural polymer chains. Examples of polymer chains include those that are formed from one or more monomers selected from the following: C₁-C₆-alkylene oxide monomers (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, 8-valerolactone, ¿-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), and 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer chains may be selected, for example, from the following polymer chains: polyether chains including poly(C₁-C₆-alkylene oxide) chains such as poly(ethylene oxide) (PEO) chains (also referred to as polyethylene glycol chains or PEG chains), poly(propylene oxide) chains, poly(ethylene oxide-co-propylene oxide) chains, polyester chains including polyglycolide chains, polylactide chains, poly(lactide-co-glycolide) chains, poly(β-propiolactone) chains, poly(β-butyrolactone) chains, poly(γ-butyrolactone) chains, poly(γ-valerolactone) chains, poly(δ-valerolactone) chains, and poly(ε-caprolactone) chains, polyoxazoline chains including poly(2-C₁-C₆-alkyl-2-oxazoline chains) such as poly(2-methyl-2-oxazoline) chains, poly(2-ethyl-2-oxazoline) chains, poly(2-propyl-2-oxazoline) chains, poly(2-isopropyl-2-oxazoline) chains, and poly(2-n-butyl-2-oxazoline) chains, poly(2-phenyl-2-oxazoline) chains, polymer chains formed from one or more polar aprotic vinyl monomers, including poly(N-vinyl pyrrolidone) chains, poly(acrylamide) chains, poly(N-methyl acrylamide) chains, poly(dimethyl acrylamide) chains, poly(N-vinylimidazole) chains, poly(4-vinylimidazole) chains, and poly(sodium 4-vinylbenzenesulfonate) chains, polydioxanone chains, poly(N-isopropylacrylamide) chains, polypeptide chains, and hydrophilic polymer chains.

Polymer chains may contain between 10 and 10000 monomer units or more.

In various embodiments, polymer chains, including those schematically represented in the preceding schemes with the letter A, a part of a multi-arm polymer where three or more polymer arms that comprise the polymer chains extend from a core region. The multi-arm polymers have three or more polymer arms (e.g., between three and fifteen polymer arms). General classes of core regions include residues of polyols, including sugars (monosaccharides, disaccharides, trisaccharides, etc.) and sugar alcohols, calixaranes, polyhedral oligomeric silsesquioxanes (POSS), cyclodextrin, polyhydroxylated polymers, catechins, flavanols, anthocyanins, stilbenes, and polyphenols, among many others.

In various embodiments, the polymer chains are crosslinked by multifunctional crosslinkers that contain two or more functional groups (e.g., between two and ten functional groups).

In some of these embodiments, the multifunctional crosslinkers are dimeric reactive linkers, and functional groups at the ends of the polymer chains are reversibly crosslinked with the dimeric reactive linkers as described above.

In some of these embodiments, the multifunctional crosslinkers have functional groups that irreversibly crosslink with functional groups at ends of the polymer chains, forming crosslinks that contain immolative linkers as described above. Two examples will be described in more detail here.

The first example provides hydrogels that having crosslinks that contain imidosydnone groups as immolative linkers. In the presence of strained alkynes, imidosyndones will rapidly undergo sydnone-alkyne click chemistry reactions.

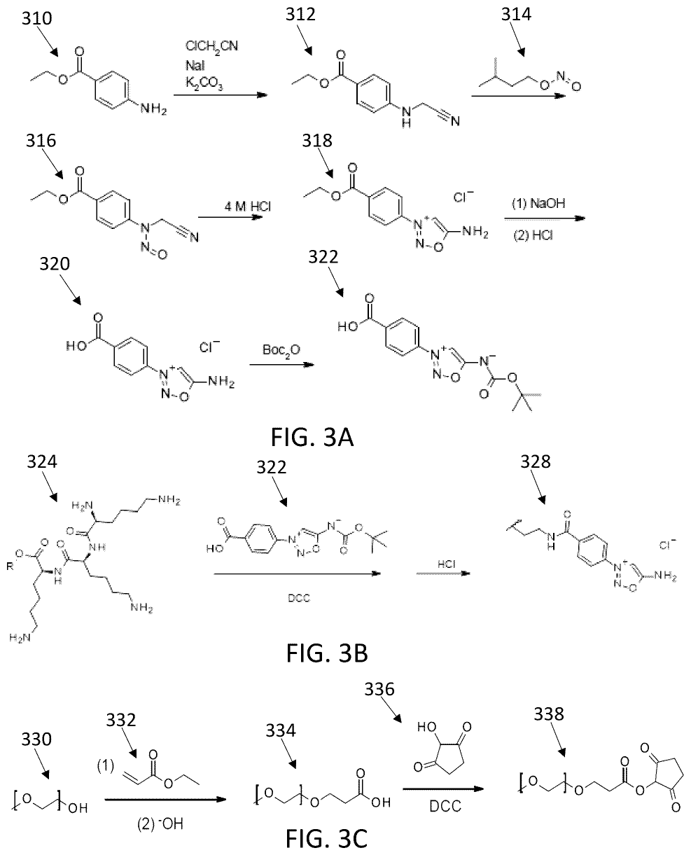

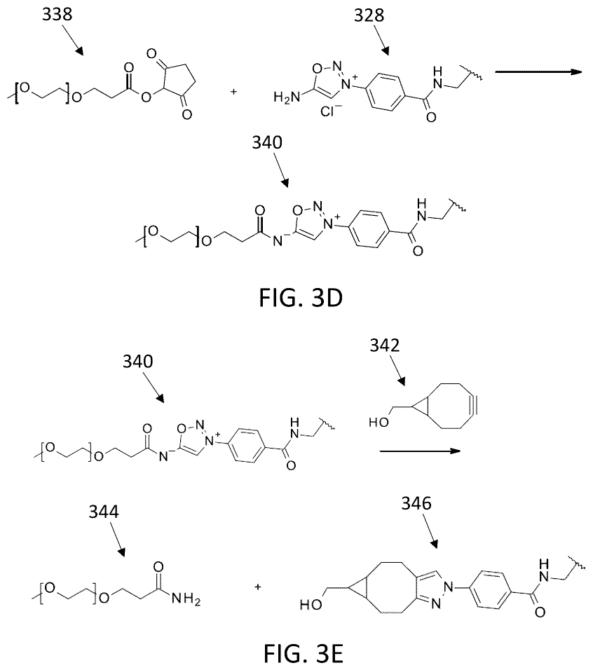

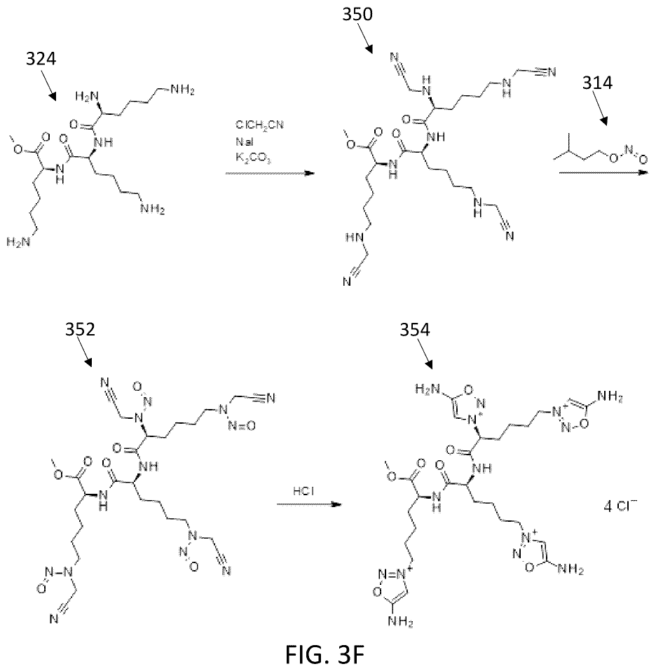

With reference now to FIG. 3A, in a first step, the amino group of 4-aminobenzoic acid methyl ester (310) is cyanomethylated with chloroacetonitrile in the presence of NaI and K₂CO₃ to yield 4-cyanomethylaminobenzoic acid methyl ester (312), which is then reacted with iso-amyl nitrite (314) to yield intermediate compound (316), followed by treatment with 4N HCl to form 4-(2-aminonitrosoethylnitrile) benzoic acid methyl ester (318), which is then sequentially treated with NaOH and HCl to form 4-imidosyndonebenzoic acid chloride (320), which is further reacted with di-tert-butyl dicarbonate (Boc₂O) to form boc-protected 4-imidosyndonebenzoic acid chloride (322).

Turning to FIG. 3B, a trilysine alkyl ester (324), where R is an alkyl group, heterocycle group, etc., that does not contain a carboxylic acid, halide, amine, alcohol, or other functional groups that can interfere with the following coupling reaction, is reacted with boc-protected 4-imidosyndonebenzoic acid methyl ester chloride (322) from FIG. 3A in the presence of a carbodiimide coupling agent such as N,N′-dicyclohexylcarbodiimide (DCC), followed by treatment in HCl to form imidosydnone-functionalized trilysine (328). Note that the terminus of only a single functionalized side chain of the three side chains of the trilysine is shown. Although trilysine is used as a multi-functional amine in this specific instance, other multi-functional amines may be used including 1,3-propanediamine, tris(3-aminopropyl)amine, 3-(2-aminoethyl) pentane-1,5-diamine, N,N′,N′-tetrakis(2-aminoethyl)-1,2-ethanediamine, 1,3,5-tris-(2-aminoethyl)-[1,3,5]triazinane-2,4,6-trione, N,N,N′-Tris(2-aminoethyl)ethylenediamine, and adamantane-1,3,5,7-tetraamine, among many others.

With reference to FIG. 3C, a hydroxyl-terminated multi-arm PEG (330) (only one arm is illustrated) is reacted with acrylic acid ethyl ester (332) followed by treatment with base to yield a carboxyl-terminated multi-arm PEG (334), which is reacted with N-hydroxy succinimide (336) in the presence of a carbodiimide coupling agent such as DCC to yield succinimidyl-ester-terminated multi-arm PEG (338). Although a hydroxyl-terminated multi-arm PEG is employed in FIG. 3C, it will be appreciated that other hydroxyl terminated multi-arm polymers may be used including those containing the polymer chains described above.

With reference to FIG. 3D, the activated succinimidyl ester groups of the succinimidyl-ester-terminated multi-arm PEG (338) of FIG. 3C can be rapidly reacted with the primary amine groups of the imidosydnone-functionalized trilysine (328) of FIG. 3B to form a crosslinked hydrogel product (340) with crosslinks that contain imidosyndoneamide linkages.

Because the resulting hydrogel contains crosslinks that comprise sydnone groups, which connect the multi-arm polymer residue and the trilysine-based crosslinker residue, when the hydrogel is no longer desired, it can be degraded on demand by treatment with a strained alkyne. In a particular example shown in FIG. 3E, the crosslinked hydrogel product (340) of FIG. 3D is contacted with a strained alkyne, in particular, (1R,8S,9s)-bicyclo[6.1.0]non-4-yn-9-ylmethanol, also known as BCN—OH, thereby breaking the crosslinks, and forming a free amido-terminated arm (344), which is associated with a residue of the incorporated multi-arm PEG, and a cyclooctapyrazole group (346), which is associated with a residue of the incorporated trilysine-based crosslinker. Although BCN—OH is used in this particular example, strained alkynes can be functionalized with different groups or added to polymers to increase their biocompatibility.

So in addition to this modification of trilysine with molecular 322, we should be able to actual modify trilysine all together with this reactive group (see below).

As an alternative to the synthesis shown in FIGS. 3A and 3B, and with reference to FIG. 3F, in a first step the amino groups of the methyl ester of trilysine (324), or amino groups of any other suitable polyamine compound, is cyanomethylated with chloroacetonitrile in the presence of NaI and K₂CO₃ to yield cyanomethylamino functionalized trilysine (350) in which the groups of the trilysine have been converted to cyanomethylamino groups. The cyanomethylamino groups of the cyanomethylamino functionalized lysine (350) are then reacted with iso-amyl nitrite (314) to a further yield intermediate compound (352), followed by treatment with 4N HCl to form imidosydnone-functionalized trilysine (328). Although trilysine is used as a multi-functional amine in this specific instance, other multi-functional amines may be used including 1,3-propanediamine, tris(3-aminopropyl)amine, 3-(2-aminoethyl) pentane-1,5-diamine, N,N′,N′-tetrakis(2-aminoethyl)-1,2-ethanediamine, 1,3,5-tris-(2-aminoethyl)-[1,3,5]triazinane-2,4,6-trione, N,N,N′-Tris(2-aminoethyl)ethylenediamine, and adamantane-1,3,5,7-tetraamine, among many others.

The second example is based on bioorthogonal click and release chemistry using a diborane.

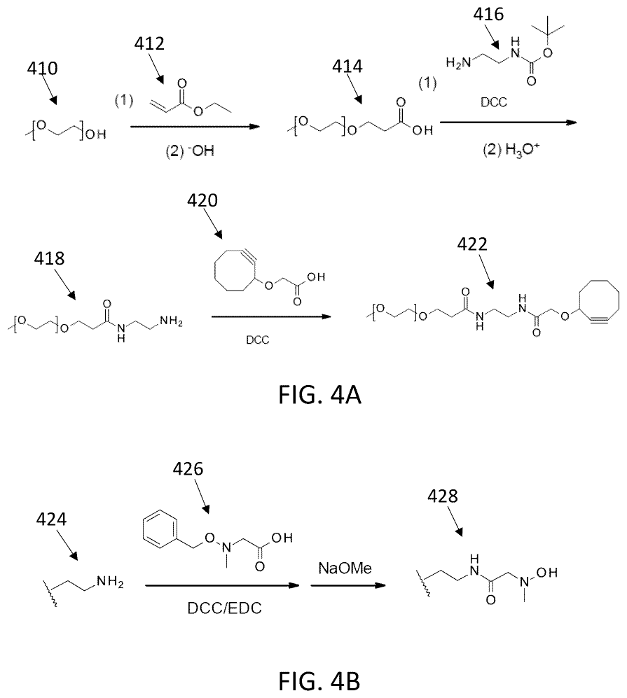

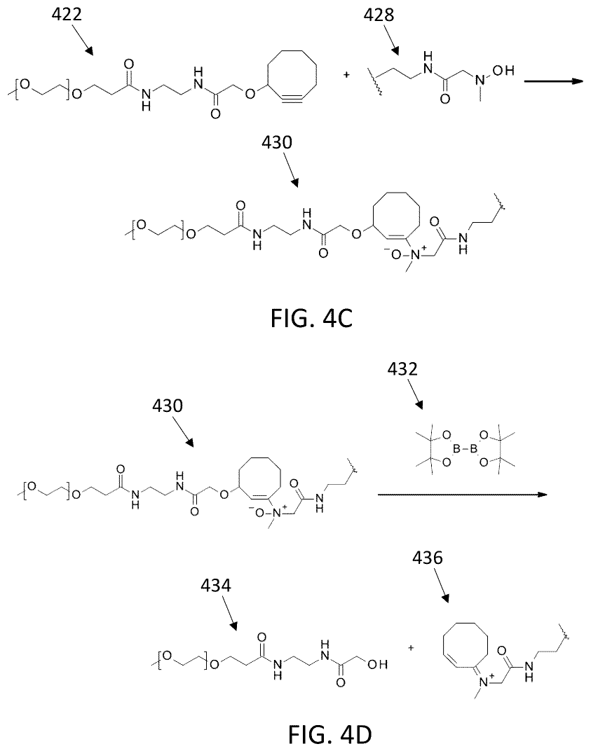

Turning to FIG. 4A, a hydroxyl-terminated multi-arm PEG (410) (only one arm is illustrated) is reacted with acrylic acid ethyl ester (412) followed by treatment with base to yield a carboxyl-terminated multi-arm PEG (414). The carboxyl-terminated multi-arm PEG (414) is reacted with N-boc-1,2-diaminoethane (416), also known as tert-butyl N-(2-aminoethyl) carbamate, in the presence of a carbodiimide coupling agent such as DCC, followed by treatment in acid to form aminoethylaminocarbonyl-terminated multi-arm PEG (418), which is reacted with 2-(cyclooct-2-yn-1-yloxy) acetic acid (420) to form ethyl(cyclooct-2-yn-1-yloxy)aminocarbonyl-terminated multi-arm PEG (422) in which the multi-arm PEG is functionalized with a strained alkyne. Although a hydroxyl-terminated multi-arm PEG is employed in FIG. 4A, it will be appreciated that other hydroxyl terminated multi-arm polymers may be used including those containing the polymer chains described above.

With reference now to FIG. 4B, trilysine (424) (the terminus of only a single aminobutyl side chain out of the three aminobutyl side chain of the trilysine is shown) is reacted with N-Methyl-N-(phenylmethoxy)glycine (426) in the presence of a carbodiimide coupling agent such as DCC, followed by reaction sodium methoxide (NaOMe) to form an N-methylhydroxylamine-functional compound (428) in which N-methylhydroxylamine groups are linked to a trilysine residue through amide linkages. Although trilysine is used as a multi-functional amine in this specific instance, other multi-functional amines may be used, including those set forth above.

With reference to FIG. 4C, the cyclooctyne groups of the cyclooctyne-terminated multi-arm PEG (422) of FIG. 4A can be rapidly reacted with the N-methylhydroxylamine groups of the N-methylhydroxylamine-functionalized trilysine (428) of FIG. 4B to form a crosslinked hydrogel product (430) with crosslinks that contain enamine N-oxide groups, specifically, 1-(methyloxidoamino) cyclooctene groups.

Because the resulting hydrogel contains crosslinks that comprise enamine N-oxide groups, which connect the multi-arm polymer residue and the trilysine-based crosslinker residue, when the hydrogel is no longer desirable, it can be degraded on demand by treatment with a diboron compound. In a particular example shown in FIG. 4D, the crosslinked hydrogel product (430) of FIG. 4C is contacted with a diboron compound, specifically, bis(pinacolato)diboron (432), thereby breaking the crosslinks and forming a free N-(2-hydroxyethyl)amide-terminated arm (434), which is associated with a residue of the incorporated multi-arm PEG, and an iminium group (436), which is associated with a residue of the incorporated trilysine-based crosslinker.

As previously indicated, present methods comprise contacting a hydrophilic polymer hydrogel, which comprises hydrophilic polymer chains that are crosslinked by crosslinks that comprise immolative linkers or by crosslinks that contain reversible covalent linkages that incorporate reactive dimeric linkers, with a cleavage composition that comprises a singly reactive molecule. For example, in the event that it is desirable to remove a hydrophilic polymer hydrogel from a subject (e.g. because therapy is complete, because the crosslinked hydrophilic polymer is causing discomfort, because the hydrophilic polymer hydrogel is improperly placed, etc.), the crosslinked hydrophilic polymer may be contacted with the cleavage composition.

In some embodiments, the cleavage composition contains a singly reactive molecule that acts to break the crosslinks at the position of the immolative linkers within the crosslinks. In some embodiments, the cleavage composition contains a singly reactive molecule that acts to break the crosslinks at the position of the dimeric linkers within the crosslinks.

In addition to one or more singly reactive molecules, the cleavage compositions of the present disclosure may contain one or more additional agents such as therapeutic agents, imaging agents, colorants, tonicity adjusting agents, suspension agents, wetting agents, and pH adjusting agents as detailed below.

In some aspects, the present disclosure provides hydrogels that comprises a reaction product of a multifunctional crosslinker and a multi-arm polymer having polymer chains that are reactive with the multifunctional crosslinker.

In some embodiments, the multifunctional crosslinker is a dimeric reactive linker, and hydrogels are formed that comprise a reaction product of the dimeric reactive linker and a multi-arm polymer wherein functional groups at the ends of hydrophilic polymer chains that form the arms of the multi-arm polymer are reversibly crosslinked with the dimeric reactive linker.

In some embodiments, hydrogels are formed that comprise a reaction product of the multifunctional crosslinker and a multi-arm polymer wherein functional groups of the multifunctional crosslinker irreversibly react with functional groups at the ends of hydrophilic polymer chains that form the arms of the multi-arm polymer, creating crosslinks that contain immolative linkers.

In some aspects of the present disclosure, systems are provided that are configured to deliver (a) a multifunctional crosslinker as described herein and (b) a multi-arm polymer as described herein. When the multifunctional crosslinker and the multi-arm polymer are comingled, crosslinks are formed between the multifunctional crosslinker and the multi-arm polymer. Such systems can be used to form hydrophilic polymer hydrogels in vivo within a subject. Such systems can also be used to form hydrophilic polymer hydrogels ex vivo, which are subsequently introduced to a subject.

In either case, once the hydrophilic polymer hydrogel is positioned within the subject, the hydrophilic polymer hydrogel may be contacted with a cleavage composition that is adapted to break down the crosslinks within the hydrophilic polymer hydrogel.

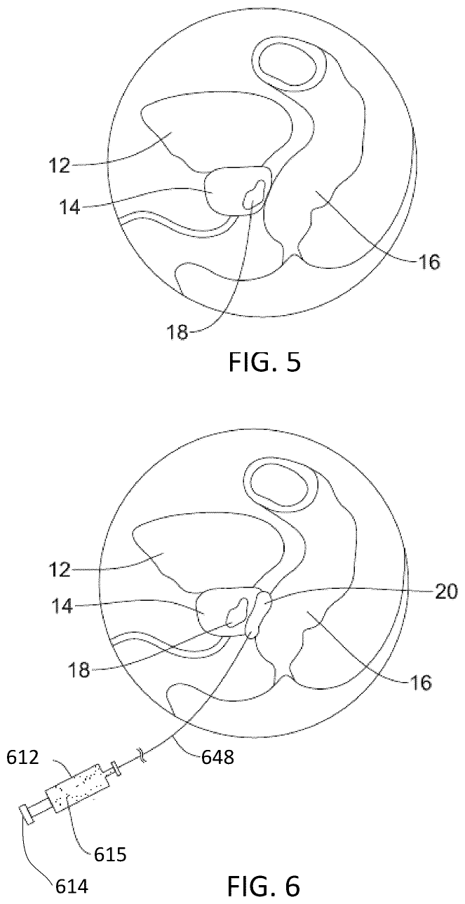

FIGS. 5-6 illustrate the placement of one such hydrophilic polymer hydrogel between the prostate and rectum of a subject. FIG. 5 is a schematic overview depicting a region of the human body including, for example, the bladder 12, prostate 14, and rectum 16. In this example, the prostate 14 may include a tumor 18. In some instances, it may be desirable to treat the tumor 18 with radiation therapy.

Prior to radiation therapy, it may be desirable to place a hydrophilic polymer hydrogel spacer 20 in the human body. As shown in FIG. 6, a preloaded syringe, in which an injectable hydrophilic polymer hydrogel composition 615 is disposed within a delivery syringe system that includes a syringe barrel 612 and a plunger 614, may be connected to a needle 648 the distal tip of which is positioned between the prostate 14 and rectum 16. The plunger 614 may be used to deliver the hydrophilic polymer hydrogel composition 615 to form a hydrophilic polymer hydrogel spacer 20. With the prostate 14 spaced from the rectum 16, radiation therapy may be used to treat the tumor 18.

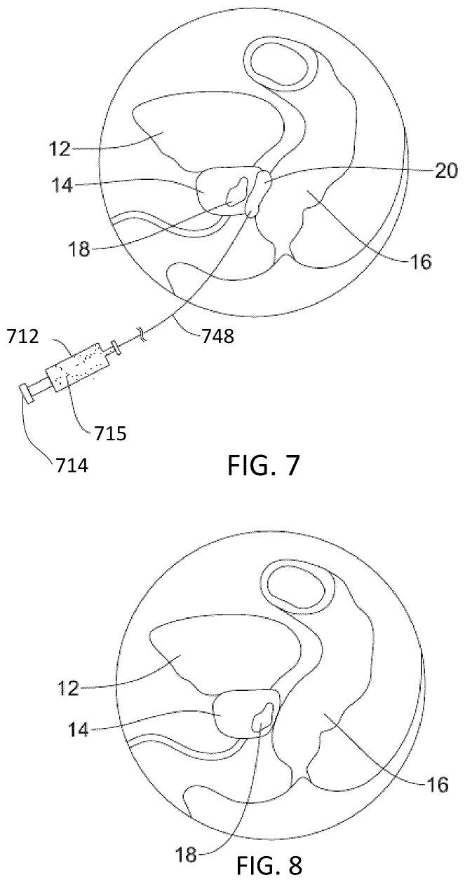

In the event that cleavage of the hydrophilic polymer hydrogel spacer 20 is desired, a cleavage composition may be contacted with the hydrophilic polymer hydrogel spacer 20 in order to accelerate cleavage of the same. As shown in FIG. 7, a preloaded syringe, in which a cleavage composition 715 is disposed within a cleavage syringe system that includes a syringe barrel 712 and a plunger 714, may be connected to a needle 748 the end of which is positioned in the hydrophilic polymer hydrogel spacer 20. The plunger 714 may be used to deliver the cleavage composition 715 to the hydrophilic polymer hydrogel spacer 20 as schematically depicted in FIG. 7. The cleavage composition 715 may act to degrade the hydrophilic polymer hydrogel spacer 20 as schematically depicted in FIG. 8.

It is noted that in some cases (e.g., wherein it is immediately apparent that the hydrophilic polymer hydrogel spacer 20 has been misplaced), the needle 748 may be the same as the needle 648 that was used to initially introduce the hydrophilic polymer hydrogel spacer 20, allowing the preloaded syringe barrel 612 to be disconnected from the needle 648 and the preloaded syringe barrel 712 to be connected to the same needle without withdrawing needle from the subject.

In the preceding embodiment, the hydrophilic polymer hydrogel is completely removed from the subject. In other embodiments, the cleavage composition is contacted with the hydrophilic polymer hydrogel in a manner such that only a portion of the crosslinks are broken, leading to softening and/or swelling of the hydrophilic polymer hydrogel in situ.

In some aspects, the present disclosure pertains to a system that comprises (a) an injectable or implantable composition comprising a pre-formed hydrophilic polymer hydrogel and (b) a cleavage composition that acts to break the crosslinks within the hydrophilic polymer hydrogel.

Various cleavage compositions are described above and include at least one singly reactive molecule. The cleavage compositions may be provided in a suitable reservoir such as a syringe, vial or ampule. Whether supplied in a syringe, vial, ampule or other reservoir, the cleavage compositions may be provided, for example, in dry form (e.g., powder form) or in fluid form, such as in a solution form, or in a multi-phase fluid such as a suspension of particles of the at least one singly reactive molecule or in an oil/water or water/oil emulsion form, wherein singly reactive molecule is predominantly present in the oil phase or the aqueous phase. The cleavage compositions may further include one or more additional agents, including therapeutic agents, imaging agents, colorants, tonicity adjusting agents, suspension agents, wetting agents, and pH adjusting agents as described below.

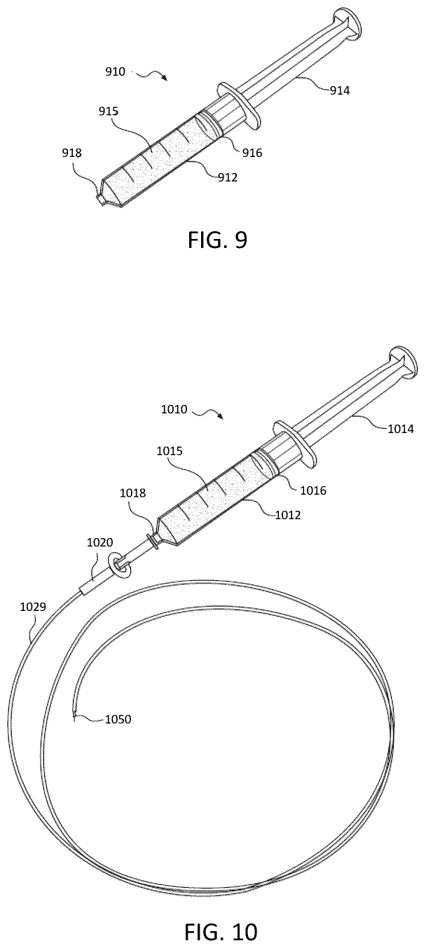

FIG. 9 illustrates a syringe 910 for injection of a cleavage composition as discussed above. The syringe 910 may comprise a barrel 912, a plunger 914, and one or more stoppers 916. The barrel 912 may include a Luer adapter (or other suitable adapter/connector) at the distal end 918 of the barrel 912, suitable for attachment to an injection needle or a flexible catheter. The syringe barrel 912 may serve as a reservoir, containing the cleavage composition 915 for injection into a subject, for example, through a needle or catheter.

Pre-formed hydrophilic polymer hydrogels may be in any desired form, including a slab, a cylinder, a coating, or particles. In some embodiments, the hydrophilic polymer hydrogel is dried and then granulated into particles of suitable size. Granulating may be by any suitable process, for instance by homogenization, forcing through a screen, grinding (including cryogrinding), crushing, milling, pounding, or the like. Sieving or other known techniques can be used to classify and fractionate the particles. Hydrophilic polymer hydrogel particles formed using the above and other techniques may varying widely in size, for example, having an average size ranging from 50 to 950 microns.

In addition to a hydrophilic polymer hydrogel as described above, preformed hydrophilic polymer hydrogel compositions in accordance with the present disclosure may contain additional agents, including therapeutic agents, imaging agents, colorants, tonicity adjusting agents, suspension agents, wetting agents, and pH adjusting agents as detailed below.

The pre-formed hydrophilic polymer hydrogel compositions may be provided in any suitable packaging. Where the hydrophilic polymer hydrogel composition is provided in injectable form (e.g., wherein the hydrophilic polymer hydrogel composition contains injectable hydrophilic polymer hydrogel particles, beads, pellets, etc.), the hydrophilic polymer hydrogel compositions may be provided in a reservoir, such as a syringe, vial or ampule. Whether supplied in a syringe, vial, ampule or other reservoir, the pre-formed hydrophilic polymer hydrogel compositions may be provided, for example, in dry form (e.g., in the form of a powder that contains hydrophilic polymer hydrogel particles) or in a fluid form (e.g., in the form of a suspension that contains hydrophilic polymer hydrogel particles).

The pre-formed hydrophilic polymer hydrogel compositions may be delivered to a subject using a suitable delivery device. Preferred subjects include mammalian subjects, particularly human subjects.

One exemplary delivery device is shown in FIG. 10, which illustrates a syringe 1010 providing a reservoir containing a pre-formed hydrophilic polymer hydrogel composition as discussed above. The syringe 1010 may comprise a barrel 1012, a plunger 1014, and one or more stoppers 1016. The barrel 1012 may include a Luer adapter (or other suitable adapter/connector), e.g., at the distal end 1018 of the barrel 1012, for attachment to an injection needle 1050 via a flexible catheter 1029. The proximal end of the catheter 1029 may include a suitable connection 1020 for receiving the barrel 1012. In other examples, the barrel 1012 may be directly coupled to the injection needle 1050. The syringe barrel 1012 may serve as a reservoir, containing a pre-formed hydrophilic polymer hydrogel composition 1015 for injection through the needle 1050.

In some embodiments, the hydrophilic polymer hydrogel compositions of the present disclosure can be imaged after administration using a suitable imaging technique such as ultrasound or an X-ray-based imaging technique, such as computerized tomography or X-ray fluoroscopy.

When desired, cleavage of the hydrophilic polymer hydrogel compositions of the present disclosure can initiated by contacting the hydrophilic polymer hydrogel compositions with a cleavage composition as described herein.

The hydrophilic polymer hydrogel compositions described herein can be used for a number of medical purposes.

For example, the hydrophilic polymer hydrogel compositions can be injected to provide spacing between tissues, the hydrophilic polymer hydrogel compositions can be injected to provide fiducial markers, the hydrophilic polymer hydrogel compositions can be injected for tissue augmentation or regeneration, including cosmetic tissue augmentation, the hydrophilic polymer hydrogel compositions can be injected as a filler or replacement for soft tissue, the hydrophilic polymer hydrogel compositions can be injected to provide mechanical support for compromised tissue, the hydrophilic polymer hydrogel compositions can be injected as a scaffold, the hydrophilic polymer hydrogel compositions can be injected as lifting agents for internal cyst removal, and/or the hydrophilic polymer hydrogel compositions can be injected as a carrier of therapeutic agents in the treatment of diseases and cancers and the repair and regeneration of tissue, among other uses. The hydrophilic polymer hydrogel compositions can also be injected into a left atrial appendage during a left atrial appendage closure procedure. In some embodiments, hydrophilic polymer hydrogel compositions may be injected into the left atrial appendage after the introduction of a closure device such as the Watchman® left atrial appendage closure device available from Boston Scientific Corporation.

The hydrophilic polymer hydrogel compositions of the present disclosure may be used in a variety of medical procedures, including the following, among others: a procedure to implant a fiducial marker comprising a hydrophilic polymer hydrogel, a procedure to implant a tissue regeneration scaffold comprising a hydrophilic polymer hydrogel, a procedure to implant a tissue support comprising a hydrophilic polymer hydrogel, a procedure to implant a tissue bulking agent comprising a hydrophilic polymer hydrogel, a procedure to implant a therapeutic-agent-containing depot comprising a hydrophilic polymer hydrogel, a tissue augmentation procedure comprising implanting a hydrophilic polymer hydrogel, a procedure to introduce a hydrophilic polymer hydrogel between a first tissue and a second tissue to space the first tissue from the second tissue.

The hydrophilic polymer hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In various embodiments, kits are provided that include one or more delivery devices for delivering a pre-formed hydrophilic polymer hydrogel composition as described herein and a cleavage composition as described herein to a subject. Such kits may include any of the following: one or more syringes, which may or may not contain the pre-formed hydrophilic polymer hydrogel composition or the cleavage composition; one or more vials, which may or may not contain the pre-formed hydrophilic polymer hydrogel composition or the cleavage composition; one or more needles (which may be compatible both with a delivery syringe system for delivering the pre-formed hydrophilic polymer hydrogel composition and a cleavage syringe system for delivering the cleavage composition); one or more flexible tubes (which may be compatible both with a syringe for delivering the pre-formed hydrophilic polymer hydrogel composition and a syringe for delivering the cleavage composition); and an injectable liquid such as water for injection, normal saline or phosphate buffered saline. Whether supplied in a syringe, vial, or other reservoir, the pre-formed hydrophilic polymer hydrogel composition and the cleavage composition may independently be provided in dry form (e.g., powder form) or in a fluid form, which may be ready for injection.

In some aspects, the present disclosure pertains to a system that comprises (a) compositions for forming, within a subject, a hydrophilic polymer hydrogel that comprises hydrophilic polymer chains and crosslinks between hydrophilic polymer chains and (b) a cleavage composition that is adapted to accelerate cleavage of the crosslinks.

As noted above, the cleavage composition may be provided in a suitable reservoir such as a syringe, vial or ampule. Whether supplied in a syringe, vial, ampule or other reservoir, the cleavage compositions may be provided, for example, in dry form (e.g., powder form) or in fluid form, such as in a solution form or in a multi-phase fluid such as a suspension of particles of the at least one singly reactive molecule or in an oil/water or water/oil emulsion form, wherein singly reactive molecule is predominantly present in the oil phase or the aqueous phase. The cleavage composition may further include one or more additional agents, including therapeutic agents, imaging agents, colorants, tonicity adjusting agents, suspension agents, wetting agents, and pH adjusting agents as described below. A particular embodiment where a cleavage composition 915 is provided within a syringe 910 is shown in FIG. 9 described above.

Compositions for forming a hydrophilic polymer hydrogel within a subject are described above and include a multifunctional crosslinker as described above and a multi-arm polymer having polymer chains that are reactive with the multifunctional crosslinker as described above (also referred to herein as a reactive multi-arm polymer).

The reactive multi-arm polymer may be provided in a suitable reservoir such as a syringe, vial or ampule. Whether supplied in a syringe, vial, ampule or other reservoir, the reactive multi-arm polymer may be provided, for example, in dry form (e.g., powder form) or in fluid form, such as in a solution form. Moreover, the reactive multi-arm polymer may further include one or more additional agents, including therapeutic agents, imaging agents, colorants, tonicity adjusting agents, suspension agents, wetting agents, and pH adjusting agents as described below.

The multifunctional crosslinker also may be provided in a suitable reservoir such as a syringe, vial or ampule. Whether supplied in a syringe, vial, ampule or other reservoir, the multifunctional crosslinker may be provided, for example, in dry form (e.g., powder form) or in fluid form, such as in a solution form. Moreover, the multifunctional crosslinker may further include one or more additional agents, including therapeutic agents, imaging agents, colorants, tonicity adjusting agents, suspension agents, wetting agents, and pH adjusting agents as described below.

In some embodiments, the reactive multi-arm polymer and the multifunctional crosslinker are simultaneously administered to the subject, after which crosslinks are formed between the reactive multi-arm polymer and the multifunctional crosslinker.

In some embodiments, a system is provided which includes a delivery device that comprises a first reservoir that contains a first fluid composition that comprises the reactive multi-arm polymer and a second reservoir that contains a second fluid composition that comprises the multifunctional crosslinker. When the first and second fluid compositions are mixed, crosslinking commences between the reactive multi-arm polymer. During operation, the first fluid composition and second fluid composition are dispensed from the first and second reservoirs and combined, whereupon the reactive multi-arm polymer crosslink with one another to form a hydrophilic polymer hydrogel in the subject.

In various embodiments, a kit is provided that includes one or more delivery devices for delivering (a) a reactive multi-arm polymer as described herein and a multifunctional crosslinker as described herein and (b) a cleavage composition as described herein to a subject. Such kits may include any of the following: a syringe, vial or other container containing the reactive multi-arm polymer; a syringe, vial or other container containing the multifunctional crosslinker; a syringe, vial or other container containing the cleavage composition; one or more needles (which may be compatible with both a cleavage syringe system for delivering the cleavage composition and a delivery syringe system for delivering the reactive multi-arm polymer and the multifunctional crosslinker); one or more flexible tubes (which may be compatible with both a cleavage syringe system for delivering the cleavage composition and a delivery syringe system for delivering the reactive multi-arm polymer and the multifunctional crosslinker); an injectable liquid such as water for injection, normal saline or phosphate buffered saline which may be provided in a syringe, vial or other container.

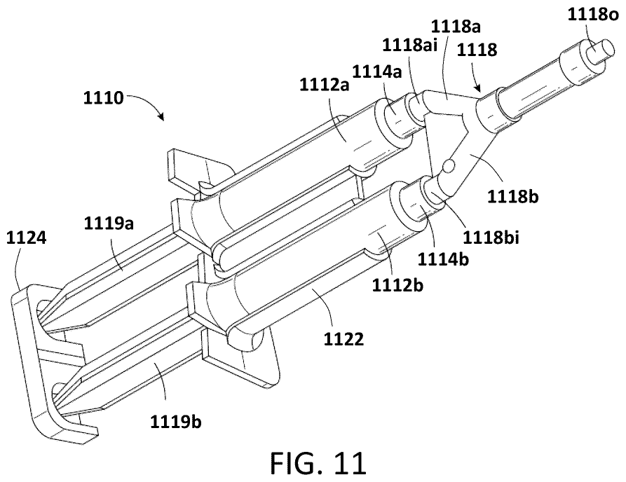

In particular embodiments, and with reference to FIG. 11, a syringe delivery system may include a delivery device 1110 that comprises a double-barrel syringe, which includes a first barrel 1112a having a first barrel outlet 1114a, which first barrel contains a first fluid composition described above, a first plunger 1119a that is movable in the first barrel 1112a, a second barrel 1112b having a second barrel outlet 1114b, which second barrel 1112b contains a second fluid composition described above, and a second plunger 1119b that is movable in the second barrel 1112b. In some embodiments, the device 1110 may further comprise a mixing section 1118 having a first mixing section inlet 1118ai in fluid communication with the first barrel outlet 1114a, a second mixing section inlet 1118bi in fluid communication with the second barrel outlet 1114b, and a mixing section outlet 11180. Also shown are a syringe holder 1122 configured to hold the first and second syringe barrels 1112a, 1112b, in a fixed relationship and a plunger cap 1124 configured to hold the first and second plungers 1119a, 1119b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject.

In either approach, a fluid admixture of the first and second fluid compositions is created which leads to the formation of a hydrophilic polymer hydrogel composition in the subject.

In some embodiments, the hydrophilic polymer hydrogel compositions of the present disclosure can be imaged after administration using a suitable imaging technique such as ultrasound or an X-ray-based imaging technique, such as computerized tomography or X-ray fluoroscopy.

In either approach, first and second fluid compositions or a fluid admixture thereof is introduced into the patient for a variety of medical purposes.

For example, the first and second fluid compositions or a fluid admixture thereof can be injected to provide spacing between tissues, the first and second fluid compositions or a fluid admixture thereof can be injected to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injection for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a hydrophilic polymer hydrogel is ultimately formed at the administration location.

After administration, the compositions of the present disclosure can be imaged using a suitable imaging technique such as ultrasound or an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a hydrophilic polymer hydrogel composition, a procedure to implant a tissue regeneration scaffold comprising a hydrophilic polymer hydrogel composition, a procedure to implant a tissue support comprising a hydrophilic polymer hydrogel composition, a procedure to implant a tissue bulking agent comprising a hydrophilic polymer hydrogel composition, a procedure to implant an embolic composition comprising a hydrophilic polymer hydrogel composition, a procedure to implant a lifting agent comprising a hydrophilic polymer hydrogel composition, a procedure to introduce a left atrial appendage closure composition comprising a hydrophilic polymer hydrogel composition, a procedure to implant a therapeutic-agent-containing depot comprising a hydrophilic polymer hydrogel composition, a tissue augmentation procedure comprising implanting a hydrophilic polymer hydrogel composition, a procedure to introduce a hydrophilic polymer hydrogel composition between a first tissue and a second tissue to space the first tissue from the second tissue.

The first and second fluid compositions or a fluid admixture thereof may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

As noted above, additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imagable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

1. A kit for delivering and degrading a hydrophilic polymer hydrogel, the kit comprising (a) a hydrogel delivery system configured to deliver a hydrophilic polymer hydrogel to a subject, the hydrophilic polymer hydrogel comprising hydrophilic polymer chains that are crosslinked by crosslinks that comprise immolative linkers or by crosslinks that contain reversible covalent linkages that incorporate a reactive dimeric linker and (b) a hydrogel cleavage system configured to deliver a cleavage composition to the subject, wherein the cleavage composition contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks.

2. The kit of claim 1, wherein the hydrogel cleavage system comprises a syringe barrel that contains the cleavage composition.

3. The kit of claim 2, wherein the hydrogel cleavage system further comprises a needle, a flexible tube, or both and wherein the cleavage syringe system is configured for coupling to the needle, the flexible tube, or both.

4. The kit of claim 1, wherein the hydrogel delivery system comprises a preformed hydrophilic polymer hydrogel.

5. The kit of claim 4, wherein the preformed hydrophilic polymer hydrogel comprises particles of the hydrophilic polymer hydrogel.

6. The kit of claim 5, wherein the particles of the hydrophilic polymer hydrogel are prepackaged in a delivery syringe system.

7. The kit of claim 1, wherein the hydrogel delivery system comprises a first composition comprising a multifunctional crosslinker and a second composition comprising a multi-arm polymer having polymer chains that are reactive with the multifunctional crosslinker.

8. The kit of claim 7, wherein the hydrogel delivery system is configured to form a mixture of the first and second compositions and to deliver the mixture to a subject whereupon the first and second compositions form said crosslinks with one another.

9. The kit of claim 7, wherein the hydrogel delivery system comprises a delivery syringe system that is configured to form a mixture of the first and second compositions and to deliver the mixture to a subject whereupon the first and second compositions form said crosslinks with one another.

10. The kit of claim 9, wherein the delivery syringe system comprises a dual barrel syringe device for creating and delivering the mixture of the first and second compositions.

11. The kit of claim 1, wherein the hydrophilic polymer hydrogel comprises the hydrophilic polymer chains that are crosslinked by crosslinks that comprise immolative linkers and wherein the hydrogel cleavage composition contains the singly reactive molecule that acts to break the crosslinks at the position of the immolative linkers within the crosslinks.

12. The kit of claim 11, wherein the crosslinks comprise imidosydnone groups as immolative linkers and wherein the singly reactive molecule is a strained alkyne reactive molecule.

13. The kit of claim 11, wherein the crosslinks comprise 1-(methyloxidoamino) cyclooctene groups as immolative linkers and wherein the singly reactive molecule is a diboron molecule.

14. The kit of claim 1, wherein the hydrophilic polymer hydrogel comprises the hydrophilic polymer chains that are crosslinked by crosslinks that contain reversible covalent linkages that incorporate a reactive dimeric linker and wherein the hydrogel cleavage composition contains the singly reactive molecule that acts to break the crosslinks at the position of the dimeric linkers within the crosslinks.

15. The kit of claim 14, wherein each of the reversible covalent linkages each comprises two thioester groups, wherein the singly reactive molecule is a thiol molecule, and wherein the reactive dimeric linker is a bis-thiol linker.

16. A kit for delivering and degrading a hydrophilic polymer hydrogel, the kit comprising (a) a hydrogel delivery system configured to deliver a preformed hydrophilic polymer hydrogel to a subject, the preformed hydrophilic polymer hydrogel comprising hydrophilic polymer chains that are crosslinked by crosslinks that comprise immolative linkers and the preformed hydrophilic polymer hydrogel comprising particles of the hydrophilic polymer hydrogel prepackaged in a syringe barrel and (b) a hydrogel cleavage system configured to deliver a cleavage composition to the subject, the cleavage composition containing a singly reactive molecule that acts to break the crosslinks at a position of the immolative linkers within the crosslinks, the cleavage composition prepackaged in a syringe barrel.

17. A method comprising (a) delivering a hydrophilic polymer hydrogel to a subject, the hydrophilic polymer hydrogel comprising hydrophilic polymer chains that are crosslinked by crosslinks that comprise immolative linkers or by crosslinks that contain reversible covalent linkages that incorporate reactive dimeric linkers and (b) delivering a cleavage composition to the subject such that the cleavage composition contacts the hydrophilic polymer hydrogel, wherein the cleavage composition contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks.

18. The method of claim 16, comprising delivering the hydrophilic polymer hydrogel to the subject via a delivery syringe system coupled to a needle; disconnecting the delivery syringe system from the needle; attaching a cleavage syringe system to the needle, the cleavage syringe system including the cleavage composition disposed in a syringe barrel; and injecting the cleavage composition into the subject.

19. The method of claim 16, further comprising observing a complication associated with the delivering of the hydrophilic polymer hydrogel to the subject prior to injecting the hydrogel cleavage material into the subject.

20. The method of claim 16, further comprising delivering a replacement hydrophilic polymer hydrogel into the subject.
